SEQUENCE LISTING (1 sequence)

SEQ ID NO: 1 - protein, 461 aa, Homo sapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

(19) United States
(12) Patent Application Publication
(10) Pub. No.: US 2013/0259854 A1
(43) Pub. Date: Oct. 3, 2013

(54) METHODS AND COMPOSITIONS FOR REDUCING THE INCIDENCE OF POST-SURGICAL ADHESIONS

(71) Applicant: SAINT LOUIS UNIVERSITY, A NON-PROFIT ORGANIZATION, St. Louis, MO (US)

(72) Inventors: Alireza R. Rezaie, Eureka, MO (US); Peyman Dinarvand, St. Louis, MO (US)

(73) Assignee: Saint Louis University, a non-profit organization, St. Louis, MO (US)

(21) Appl. No.: 13/854,117

(22) Filed: Mar. 31, 2013

Related U.S. Application Data
(60) Provisional application No. 61/619,257, filed on Apr. 2, 2012.

(51) Int. Cl. A61K 38/48

(52) U.S. Cl. CPC A61K 38/4866
USPC 424/94.64

(57) ABSTRACT

The disclosure relates to a method of reducing the incidence of post-surgical adhesions in a subject undergoing surgery. More specifically, the method relates to reducing the incidence of post-surgical adhesions with the topically administration of activated protein C (APC) to the internal organs and tissues exposed and/or manipulated during surgery.

### CROSS REFERENCE TO RELATED APPLICATIONS

This application claims priority to provisional application 61/619,257, filed Apr. 2, 2012, which is hereby incorporated by reference in its entirety.

### GOVERNMENT FUNDING

This work was supported by National Institutes of Health grant no. HL 101917. The government of the United States may have certain rights in this invention.

### FIELD OF THE INVENTION

[The invention relates to methods and compositions, for reducing the incidence of post-surgical adhesions, or post-surgical band formation resulting from general, abdominal, pelvic or cardiac surgery.

### BACKGROUND

Post-surgical adhesions are pathological fibrous bands which form between organs and tissues after surgery. Post-surgical adhesions due to abdominopelvic surgery, also known as peritoneal adhesions, are pathological fibrous bands which join intra-abdomen and intra-pelvic organs to each other and to peritoneal wall. Post-surgical adhesions may increase in strength with time, sometimes causing problems years after surgery. Post-surgical adhesions are also associated with high mortality and morbidity including intestinal obstruction, pelvic pain, and female infertility. Post-surgical adhesions are the most frequent result of abdominopelvic and cardiac surgery. Anyone undergoing abdominopelvic or cardiac surgery runs the risk of developing post-surgical adhesions; however, the risk is greater after operations on the lower abdomen and pelvis, including bowel and gynecological surgeries. Adhesion bands occur in 67% to 93% of patients undergoing general surgical operations and up to 97% of patients undergoing open gynecological pelvic surgeries (Liakakos et al, (2001) Dig Surg, 18:260-73; Menzies (1990) Ann R Coll Surg Engl, 72:60-3). Post-surgical adhesions are the most important causes of intestinal obstruction (Menzies (1992) Surgery annual, 24:27), pelvic pain (Sulaiman et al., (2000) The Journal of Pathology, 192:396-403), female infertility (Kavic (2002) JSLS, 6:99-109) and adhesion of heart to surrounding tissues (Seeger et al., (1997) Journal of Surgical Research, 68:63-66). Post-surgical adhesions are believed to be more common after surgical trauma, including mild surgical trauma of internal tissues and organs. Surgical trauma may include incisions, manipulations, surface desiccation, or the contact of the internal tissues with foreign materials, such as gauze, surgical gloves, or stitches. Attempts to reduce post-surgical adhesions through gentle tissue handling, meticulous hemostasis, or avoiding desiccation and contamination of the abdominal or pelvic cavity with foreign materials through the use of starch-free gloves, lint-free gauze and absorbable sutures have been attempted (Holmdahl et al. (1997) Eur J Surg Suppl, 56-62.). However it is believed that superior surgical technique may only provide marginal improvement. The focus is now to develop methods and compositions which may be administrated at the time of surgery.

Anti-inflammatory agents, antibiotics, fibrinolytic agents and solid barriers have been used for the prevention of post-surgical adhesions (Hemadeh et al., (1993) Surgery, 114:907-10; Hellebrekers et al., (2000) Hum Reprod, 15:1358-63; Kusunoki et al., (2005) Surg Today, 35:940-5; Becker et al., (1996) J Am Coll Surg, 183:297-306; Dinarvand et al., (2012) J Surg Res, 172:e1-9); (Seeger et al., (1997) Journal of Surgical Research, 68:63-66). For example, inflammation may be reduced by the administration of drugs such as corticosteroids and non-steroidal anti-inflammatory drugs. The removal of fibrin deposits has been investigated using proteolytic and fibrinolytic enzymes, but these results have been unsatisfactory. Most successful have been the use of physical barriers, which physically prevent adjacent tissues from contacting each other and thereby reducing the probability that band formation will occur. Examples of barrier materials include films such as those formed from oxidized regenerated cellulose (e.g., Interceed™, Gynecare, Ethicon division of Johnson and Johnson, Arlington, Tex., USA), hyaluronate/carboxymethylcellulose (Seprafilm™, Genzyme Corporation, Cambridge, Mass.) and polytetrafluoroethylene (Preclude™, W.L. Gore & Associates, Flagstaff, Ariz., USA), among others. Selected agents including sodium hyaluronate/carboxymethylcellulose (HA/CMC) (Seprafilm) and oxidized regenerated cellulose (Interceed) have been approved by the FDA and are the gold standards for the prevention of adhesion bands. However, a limitation of using physical barriers is their site-specific nature, which requires the surgeon to predict where adhesions may occur, and to place these barriers accordingly.

There has been no satisfactory method of treatment for post-surgical adhesions. There exists a long felt need for a treatment that is effective and easy to apply. The Inventors have discovered that the topical administration of a low dose of activated protein C (APC) to the internal organs and surrounding tissues of the peritoneal, after the primary surgery or surgical manipulation is performed, but prior to closing of the major abdominopelvic incision, will produce a significantly better result than the currently accepted methods such as the use of Seprafilm for prevention of post-surgical adhesions. It is believed that this method may be applied generally to all surgeries where post-surgical adhesions occur.

### SUMMARY OF THE INVENTION

A method of reducing the incidence of post-surgical adhesions in a subject undergoing surgery, the method comprising, topically administering an effective amount of activated protein C to internal organs and tissues exposed by surgery or subjected to surgical manipulation, prior to closing of the major incision

### REFERENCE TO COLOR FIGURES

The application file contains at least one figure executed in color. Copies of this patent application publication with color photographs will be provided by the Office upon request and payment of the necessary fee.

### DESCRIPTION OF THE FIGURES

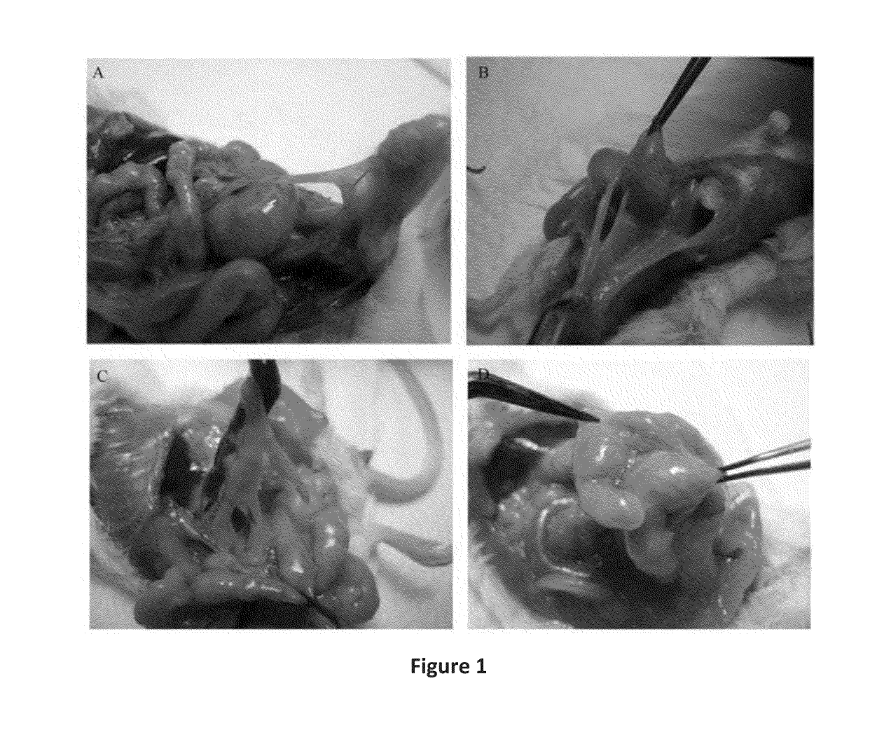

FIG. 1 illustrates intraperitoneal adhesion formation in mice. (A) Grade 1, only one band of adhesion between one viscera and the abdominal wall (Nair et al scale). A filmy thickness, Avascular bands (Leach et al scale). (B) Grade 2, two adhesion bands from viscera to abdominal wall (Nair et al scale). Limited vascularity, moderate thickness (Leach et al). (C) Grade 3, More than two bands between viscera or viscera to abdominal wall, or whole of intestines forming a mass without adherent to abdominal wall (Nair et al scale). (D) Grade 4, viscera directly adherent to abdominal wall (Nair et al). Grade 3, well vascularized, dense thickness (Leach et al scale).

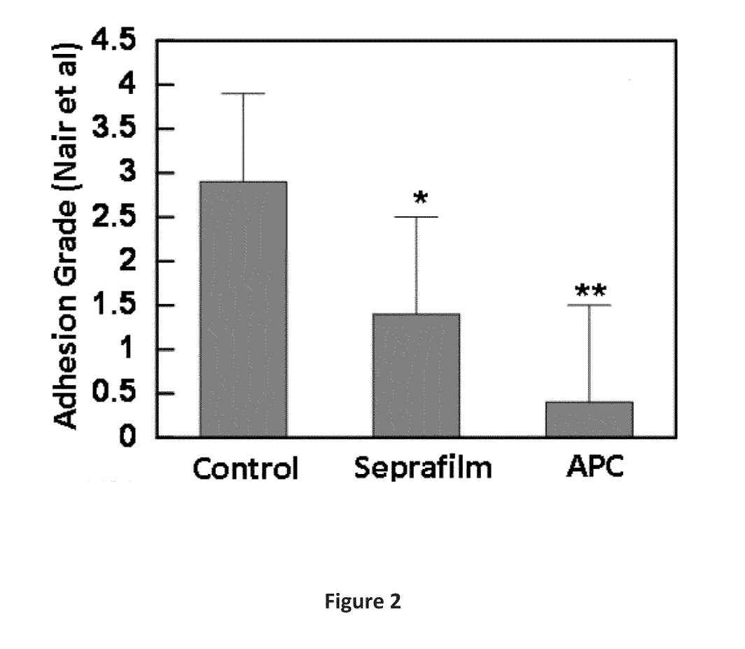

FIG. 2 shows adhesion scores of each group explained by Nair et al; the data are shown as mean±SD. A significant difference (P<0.05) was shown between the groups with (*) and all other groups; (**) and the control and Seprafilm groups.

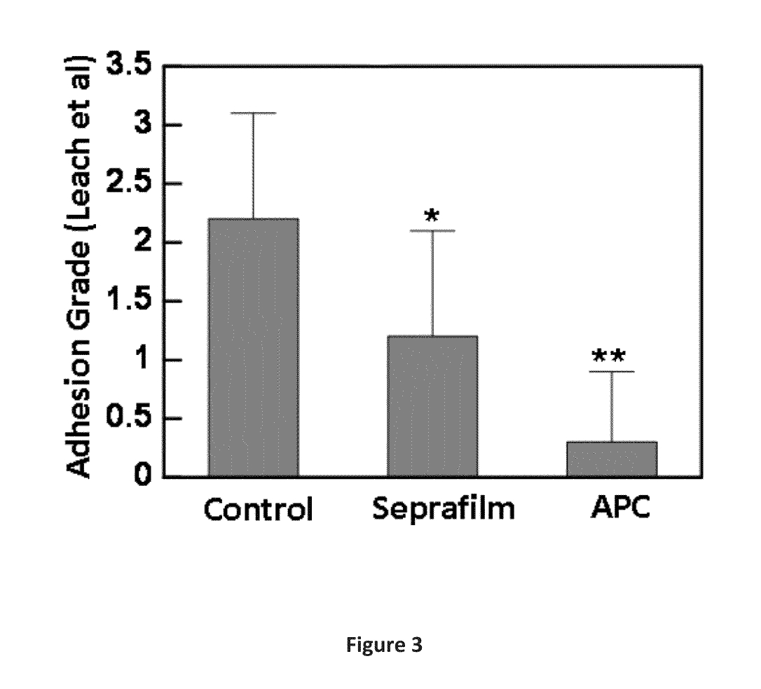

FIG. 3 shows adhesion scores of each group explained by Leach et al; the data are shown as mean±SD. The significant difference (P<0.05) has been shown between the groups with (*) and all other groups; (**) and the control and Seprafilm groups.

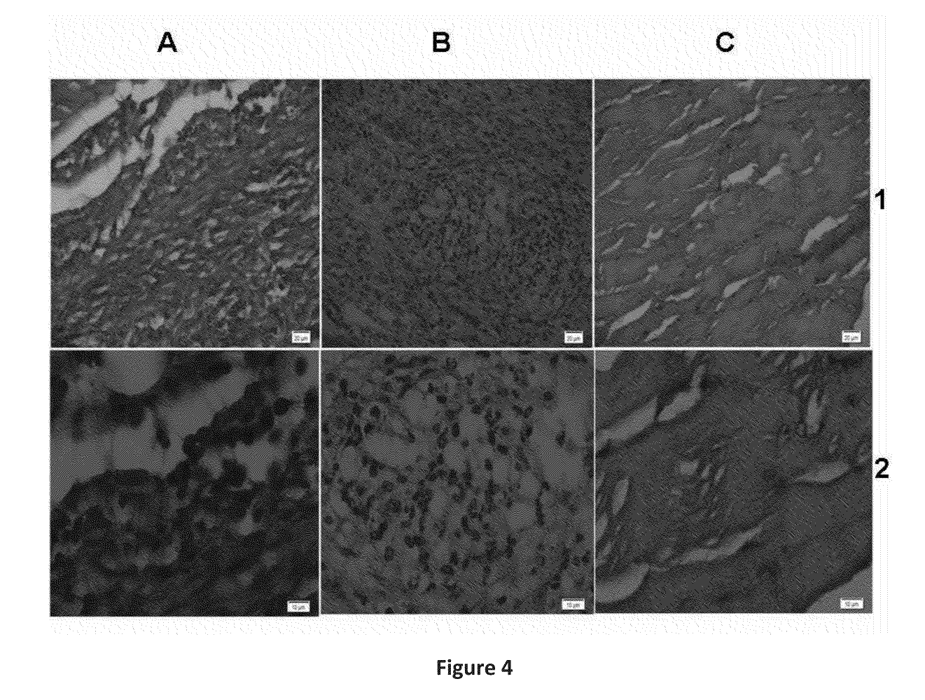

FIG. 4 shows representative images of peritoneal tissue histology in two different magnifications. The untreated control group is shown in panels A1 and A2; the Seprafilm group is shown in panels B1 and B2 and the APC group is shown in panels C1 and C2. Numerous inflammatory cells were recruited to peritoneal tissues in both control and Seprafilm groups. Only a few scattered inflammatory cells were found in the APC group.

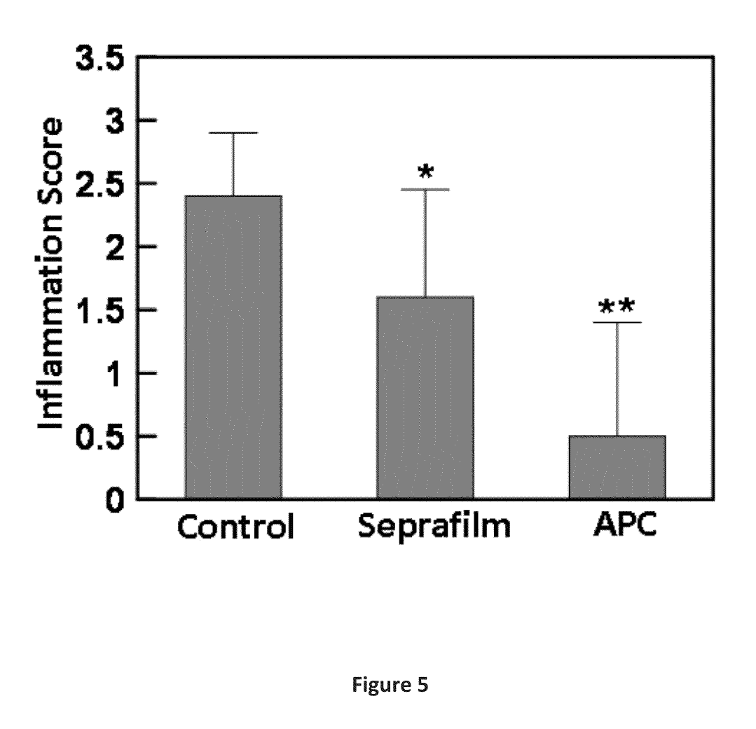

FIG. 5 shows inflammation scores of each group; the data are shown as mean±SD. The significant difference (P<0.05) has been shown between the groups with (*) and all other groups; (**) and the control and Seprafilm groups.

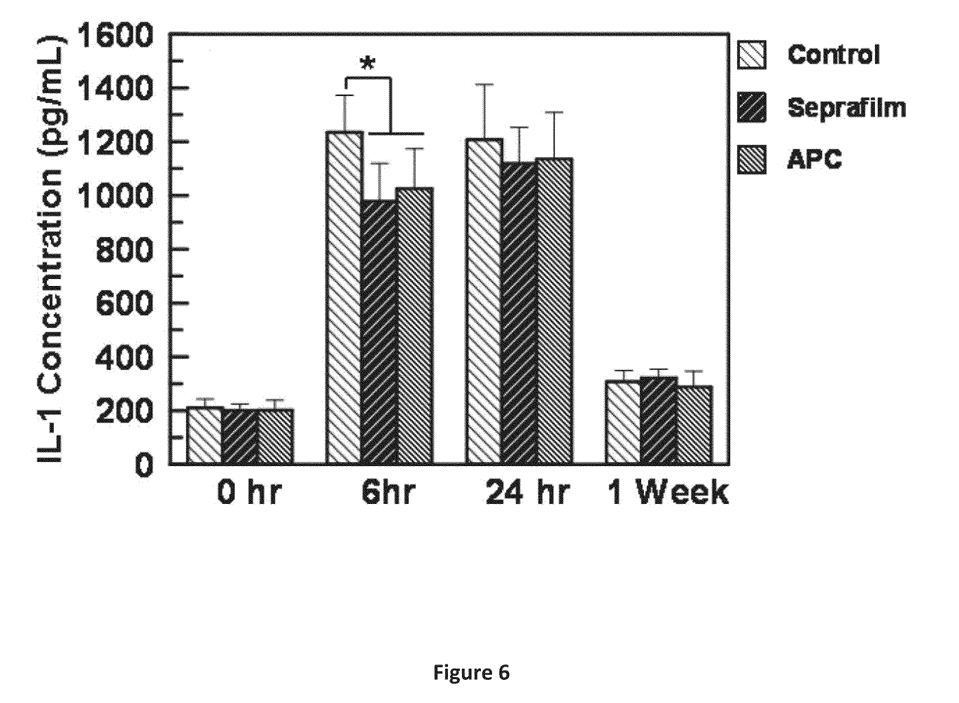

FIG. 6 shows peritoneal fluid concentrations of IL-1 in different groups; the results are shown as mean±SD. The significant difference (P<0.05) has been shown between the groups with (*) and the control.

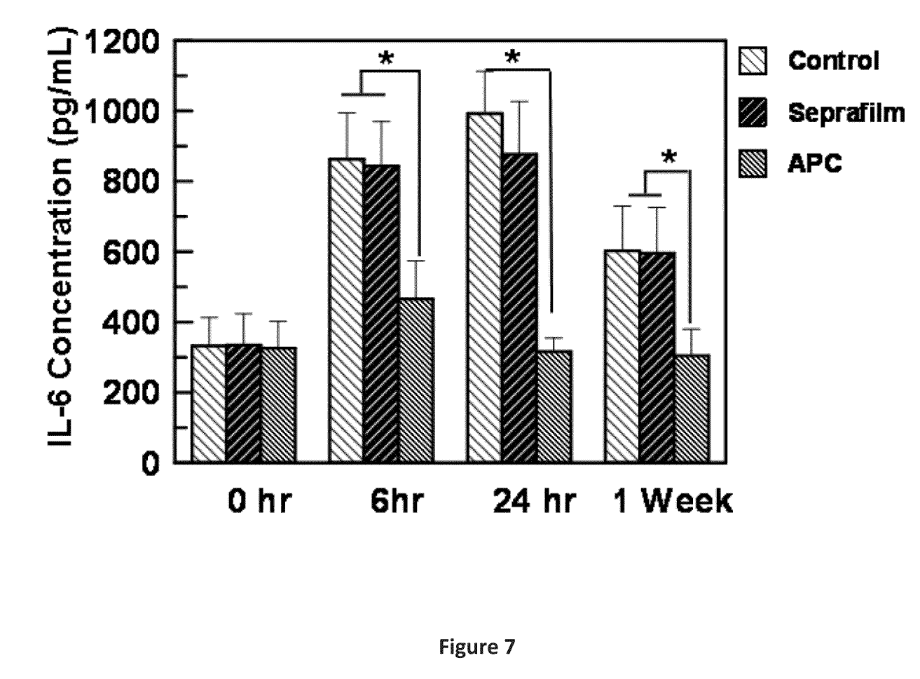

FIG. 7 shows peritoneal fluid concentrations of IL-6 in different groups; the results are shown as mean±SD. The significant difference (P<0.05) has been shown between the group with (*) and the groups of Seprafilm and control.

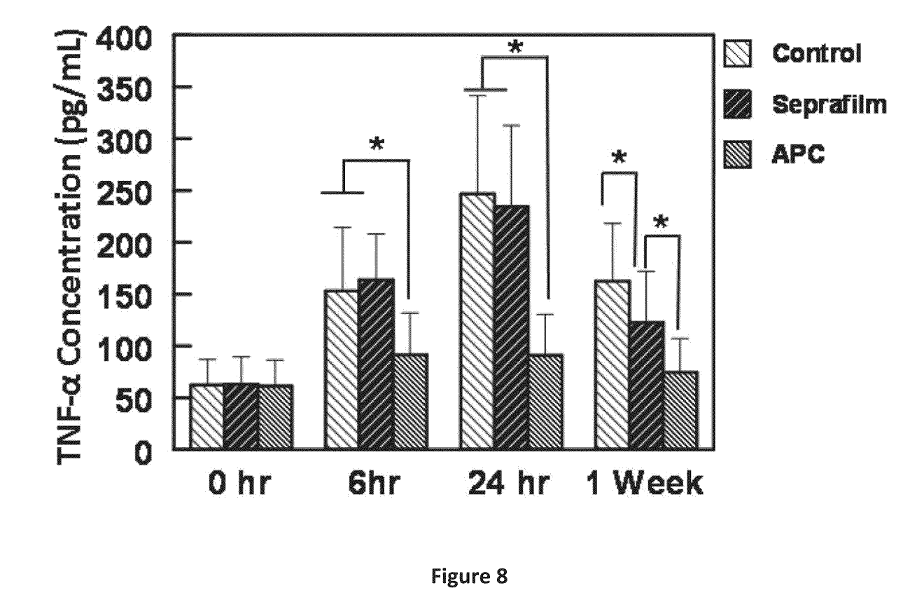

FIG. 8 shows peritoneal fluid concentrations of TNF-α in different groups; the results are shown as mean±SD. The significant difference (P<0.05) has been shown between the groups with (*) and other groups.

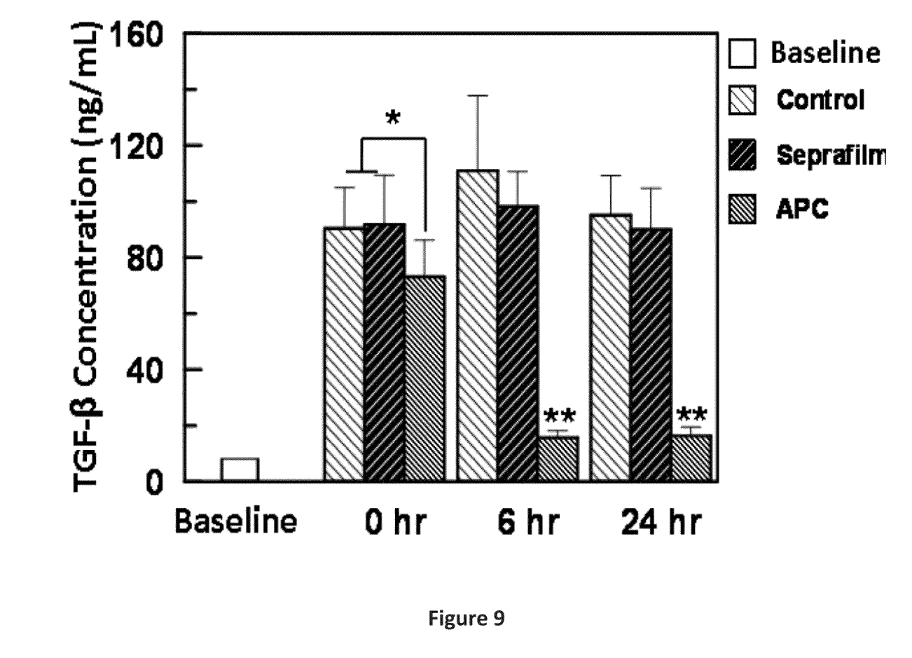

FIG. 9 shows peritoneal fluid concentrations of TGF-β in different groups. The concentrations of TGF-β (panel B) were measured by ELISA. The data are shown as mean±SD. *p<0.05 as compared to untreated control or both control and Seprafilm groups in panel A. *p<0.05 and **p<0.001 as compared to both untreated control and Seprafilm groups in panel B.

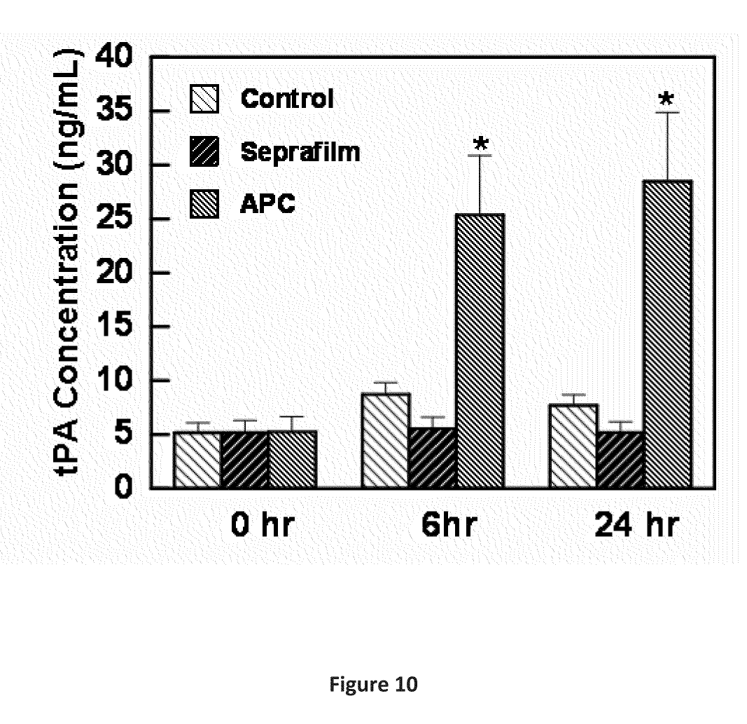

FIG. 10 shows peritoneal fluid concentrations of tPA in different groups. The tPA concentration was measured by an ELISA. The data are shown as mean±SD. *p<0.001 as compared to both untreated control and Seprafilm in each group.

### DETAILED DESCRIPTION OF THE INVENTION

The invention provides a novel approach for preventing, inhibiting, treating, or reducing the incidence of post-surgical adhesions that may form between internal organs and tissues following general, abdominal, pelvic, abdominopelvic, or cardiac surgery.

Without wishing to be bound by theory, it is believed that post-surgical adhesions begin to form within hours or days after surgery. These may arise from mild surgical trauma such as a local abrasion, incision, or even the temporary drying of the surface of some organs or tissues, which may occur despite the surgeon's best efforts. Even mild trauma may result in inflammation which may release various inflammatory factors and/or cytokines. This may in turn cause increased vascular permeability, fibrinogen activation, and fibrin deposition. A fibrin matrix may become established between abutting organs and tissues, and the lining of the abdominal, pelvic, or chest cavity. The fibrin matrix may in turn become infiltrated with fibroblasts, which may in turn deposit collagen into the matrix, thereby fortifying the matrix and possibly inducing angiogenesis. The Inventors reasoned that if this cascade of events can be reduced or prevented within the first few hours or days following surgery then subsequent matrix and adhesion band formation may be reduced or eliminated. The Inventors reasoned that activated protein C (APC), a natural anticoagulant and anti-inflammatory serine protease found in plasma may be effective if applied locally to the organs and tissues of the abdominal, pelvic, or chest cavity, following the surgical events that may result in trauma, in order to prevent these early stages of post-surgical adhesion formation.

The Inventors reasoned that a topical application of APC intraperitoneally, or on sites of surgical trauma, may inhibit inflammatory events, the deposition of fibrin, or both, and reduce the incident of post-surgical band formation at a later date. To this end the Inventors devised a treatment method whereby APC is applied topically to organs exposed through surgery, as well as generally to the abdominal, pelvic, or chest cavity, prior to closing of the major abdominal, pelvic, or chest incision. The topical application of APC, directly to sites with the potential for inflammation and fibrin deposition will also reduce the total amount of APC administered and possibly reduce collateral effects that may be caused by APC when administered systemically. The most preferred effective amount of 50 micrograms of APC, per kilogram body weight of the subject, is relatively low compared to dosages that have been used in the treatment of sepsis. Topical application is easily achieved in the operating room by surgeons.

Therefore, one embodiment of the invention is a method of reducing the incidence of post-surgical adhesions by bathing the internal organs and tissues in an adequate volume of an aqueous solution containing an effective amount of activated protein C (APC). The internal organs and tissues to be treated are those that have been exposed by, or have undergone, surgery, as well as adjacent organs and tissues. Preferably, the application of an aqueous solution containing an effective amount of APC is applied subsequent to surgery on, or manipulation of, the internal organs and tissues, and prior to closing the major incision. This surgery on, or manipulation of, the internal organs and tissues, is also referred to herein as the primary surgery. The incision that opens the body cavity providing access to the internal organs and tissues is referred to herein as the major incision. An adequate volume will be determined by the surgeon as that volume necessary to bath the exposed organs and tissues, which will vary depending on the nature of surgery and size of the subject. By way of example, in anticipation of abdominal, pelvic, or chest surgery, a surgeon may determine an adequate volume required to bath the internal organs and tissues of the abdominal, pelvic, or chest cavity. The effective amount is determined based on the subject's body weight and dissolved or diluted in the adequate volume of aqueous solution prior to application. After completion of the primary surgery, the surgeon applies the adequate volume of aqueous solution containing the effective amount of APC onto the surface of the exposed organs and tissues, as well as generally to the surrounding the abdominal, pelvic, or chest cavity. By way of example, the surgeon may use a syringe or similar device as an applicator to apply an adequate volume of aqueous solution containing APC onto the organs and tissues in the abdomen, pelvic, or chest region, before closing the abdominal, pelvic, or chest wall with sutures. The solution containing APC, once applied, may be removed from the subject, but is preferably left in place. Care may be taken by the surgeon to treat all exposed organs, particularly any organs which may have received mild or significant trauma.

Post-surgical adhesions are associated with high mortality and morbidity including intestinal obstruction, pelvic pain, and female infertility. An application of APC as described herein may reduce the incidence of these events. It is also expected that there may be a reduction in the inflammatory events associated with post-surgical adhesions. Non-limiting examples of some indicators of inflammatory events include IL-1, IL-6, TNF-α, TGF-β, or tPA. As described in the examples, these indicators may be measured in any of the appropriate bodily fluid associated with the surgery, particularly in the peritoneal fluid, as possible indicators of abdominal or pelvic abdominopelvic post-surgical adhesions. As demonstrated in the examples it is expected that a reduction in these indicators may represent a reduction in the incidence of post-surgical adhesions.

### I. Activated Protein C

Human protein C is the inactive zymogen form of a vitamin K-dependent plasma serine protease. Protein C, in vivo, or as secreted by a eukaryotic cell in culture, exists in the form of a disulfide-linked two chain molecule. It is transcribed as a single polypeptide, (see SEQ ID NO:1), which then undergoes post-transitional modification. Modifications include removal of a signal peptide sequence (amino acids 1-42), and removal of a dipeptide sequence (amino acids 198-199), which produces two polypeptides referred to as the light (˜25 kD) (amino acids 43-197) and heavy chains (˜41 kD) (amino acids 200-461). Variations in molecular weight occur due to differences in glycosylation, which is also a post-translational modification. The light chain contains a region of gamma-carboxyglutamic acid, which is required for membrane binding and is dependent on Ca²⁺. The heavy chain contains the serine protease domain, which also contains a Ca²⁺ binding site described in detail below. The heavy chain also contains the activation peptide. Activation of protein C to activated protein C takes place in vivo by removal of this activation peptide (amino acids 200-211) by thrombin. A disulfide bond at cysteine 183 and cysteine 319 connects the heavy and light chains. (Plutzky et al., (1986) Proc. Natl. Acad. Sci. (USA) 83, 546-550)

### i) Activation and Anticoagulation Activity

In vivo, protein C circulates as an inactive zymogen. Activation of protein C to activated protein C takes place by proteolytic removal of the activation peptide (amino acids 200-211, see SEQ ID NO:1), from the heavy chain. Protein C is activated on the surface of endothelial cells by a thrombin-thrombomodulin (thrombin-TM) complex, which is also accelerated by the endothelial protein C receptor (EPCR). This is believed to take place by co-locating protein C with the thrombin-TM complex on the endothelial cell surface (Stearns-Kurosawa et al. (1996) Proc Natl Acad Sci. (USA); 93:10212-10216). After activation, activated protein C down-regulates the clotting cascade via a feedback loop mechanism (Stenflo J. (1984) Thromb Hemost. 10:109-121; Esmon CT. (1993) Thromb. Haemost. 70.1-5). Once protein C is activated it may dissociate from EPCR, and form a complex with the vitamin K-dependent protein cofactor, protein S. This complex will shut down the generation of thrombin derived from the cofactor effect of factors Va (Na) and VIIIa, which are known to be procoagulant cofactors of the prothrombinase and intrinsic Xase complexes, respectively.

### ii) Anti-Inflammatory and Cytoprotective Properties

In addition to providing anti-coagulant activity, APC has anti-inflammatory and anti-apoptotic proprieties. When APC is associated with EPCR, it elicits protective signaling responses in endothelial cells (Taylor et al. (1987) J Clin Invest. 1987; 79:918-925; Taylor et al. (2000) Blood; 95:1680-1686; Joyce et al. (2001) J Biol Chem.; 276:11199-11203; Ruf et al. (2003) J Thromb Haemost. 1:1495-1503; Mosnier et al. (2004) Blood. 104:1740-1744; Finigan et al. (2005) J Biol Chem.; 280:17286-17293). These protective signals may account for the beneficial effects associated with APC when used as an anti-inflammatory agent for treating severe sepsis patients (Bernard et al. (2001) N Eng J. Med.; 344:699-709). The mechanisms of the anti-inflammatory and cytoprotective effects of APC are not well understood, however, it is believed that an APC/EPCR complex cleaves protease-activated receptor-1 (PAR-1) to initiate protective signaling events in endothelial cells (Ruf et al. (2003) J Thromb Haemost. 1:1495-1503; Mosnier et al. (2004) Blood. 104:1740-1744). PAR-1 cleavage by APC may also be required for the inhibition of apoptosis in human brain endothelial cells induced by hypoxia (Cheng et al. (2003) Nature Med.; 9:338-342).

iii) Anticoagulant and Anti-Inflammatory Activity and Cytoprotective Properties

The mechanism through which protein C, once activated, functions in the anti-coagulant pathway has been extensively studied and is well understood (Walker et al. (1992) FASEB J; 6:2561-2567). After activation, APC may dissociate, from EPCR and bind to protein S, where it functions as an anticoagulant by degrading factors Va and VIIIa. Specific recognition of procoagulant factors Va and VIIIa, is determined by the basic residues of an APC exosite (Friedrich et al. (2001) J Biol Chem.; 276:23105-23108; Manithody et al. (2003) 101:4802-4807; Gale et al. (2002) J Biol Chem.; 277:28836-28840). These basic residues are clustered on three exposed surface loops referred to as 37-39 loop, 60-68 loop and 70-80 loop (chymotrypsin numbering system) (Bode et al. (1989) EMBO J.; 8:3467-3475). These basic residues constitute a binding site for TM in the thrombin-TM complex. With the exception of the 60 loop, they are also involved in recognition and subsequent degradation of factors Va and VIIIa by APC in the anti-coagulant pathway (Friedrich et al. (2001) J Biol Chem.; 276:23105-23108; Manithody et al. (2003) Blood; 101:4802-4807; Gale et al. (2002) J Biol Chem.; 277:28836-28840).

Activated protein C (APC) as referred to herein, refers to any naturally occurring APC, or recombinant produced APC, preferably but not limited to human APC. Also referred to herein, are derivatives of APC having full or substantially full protein C proteolytic, amidolytic, esterolytic, and biological (anticoagulant and/or pro-fibrinolytic) activities. Examples of protein C derivatives are described by Gerlitz, et al., U.S. Pat. No. 5,453,373, and Foster, et al., U.S. Pat. No. 5,516,650, the entire teachings of which are hereby included by reference. Recombinant activated protein C may be produced by activating recombinant human protein C zymogen in vitro or by direct secretion of the activated form of protein C. Protein C may be produced in prokaryotic cells, eukaryotic cells, transgenic animals, transgenic plants, or gene therapy, including, for example, secretion from human kidney 293 cells as a zymogen then purified and activated by techniques known to the skilled artisan. Also included are commercially available forms of APC, an example of which, commonly goes by the trade name Xigris, and is generically known as Drotrecogin Alfa (Activated), available from Eli Lilly & Co., Indianapolis, Ind. A non-limiting example of a human APC is represented by SEQ ID NO:1. The polypeptide set forth in SEQ ID NO:1, when transcribed and secreted by a eukaryotic cell, and therefore subjected to the post-translational modifications described above, to produce human protein C, may be further activated by thrombin to generate human activated protein C. It is also expected that mutants and variants of this human protein C or activated protein C, with conservative amino acids substitutions, that retain protein C or activated protein C activity, may also be effective in preventing post-surgical adhesions when applied as described herein.

### II. Formulations and Administration of Activated Protein C (APC)

### A. Pharmaceutical Dosage Form

Pharmaceutically acceptable formulations are well known for use as injectable solutions of APC. It is expected that most known injectable physiological solutions or formulations will be acceptable for the topical peritoneal application described herein. However, the amounts of APC per milliliter of solution may vary widely. By way of example, an effective amount of APC which may be obtained commercially as a sterile solution and diluted into a larger volume, or adequate volume, of sterile aqueous solution to be applied as described herein.

APC is a hydrophilic polypeptide and may be administered in a sterile aqueous solution, preferably in a physiological solution. A physiological solution may be comprised of isotonic balanced salts with a pH of about 7.0 to about 7.5. A preferred physiological solution may comprise isotonic saline and a pH of 7.5.

The aqueous solution may further contain various salts or buffers that are well known in the art. Injectable preparations, for example, sterile aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent. Non-limiting examples of acceptable vehicles and solvents that may be employed include, Ringer's solution, or isotonic sodium chloride solution.

It is preferable to maintain the pH in a physiological range, from about 6.0 to about 6.5, or about 6.5 to about 7.0, or about 7.0 to about 7.5, or about 7.5, to about 8.0, or most preferably about 7.0 to about 7.5. To maintain effective pH control, the activated protein C solution should contain a pharmaceutically acceptable buffer.

Similarly, it is preferable to maintain the ionic strength in a physiological range. The ionic strength is generally determined by the salt concentration of the solution. Pharmaceutically acceptable salts typically used to generate ionic strength include, but are not limited to, potassium chloride (KCl) and sodium chloride (NaCl). The preferred salt is maintained in a physiological range; for example, sodium chloride may be used at a concentration of 0.9 percent by weight or 100 to 150 mM.

Formulations developed for activated protein C are also known in the art and including those described in U.S. Pat. Nos., 6,630,137, 6,159,468, and 6,359,270 which are hereby incorporated by reference. Activated protein C may be formulated to prepare a pharmaceutical composition comprising as the active agent, protein C or activated protein C, and a pharmaceutically acceptable solid or carrier. For example, a desired formulation would be one comprising a bulking agent such as sucrose, a salt such as sodium chloride, a buffer such as sodium citrate and activated protein C. Formulations may be lyophilized for storage, and hydrated before use. Examples of stable lyophilized formulations include 5.0 mg/ml activated protein C, 30 mg/ml sucrose, 38 mg/ml NaCl and 7.56 mg/vial citrate, pH 6.0; and, 20 mg/vial activated protein C, 120 mg/ml sucrose, 152 mg/vial NaCl, 30.2 mg/vial citrate, pH 6.0.

Alternatively, APC formulated into pharmaceutical compositions are administered by a number of different means that will deliver a therapeutically effective dose. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

### B. Administration of Therapeutically Effective Amounts

A therapeutically effective amount, also referred to herein as an effective amount, of APC may be determined by a skilled practitioner, typically a medical doctor, and typically based on based on a subject's weight. The skilled practitioner may also monitor post-surgical events in the same or similar subjects and adjust the effective amount in subsequent surgeries. Examples of post-surgical events that may be monitored include levels of the inflammatory indicators as described herein, namely Il-1, Il-6, TNFα, and TGFβ in addition to tPA. Inflammatory indicators are preferably monitored in the appropriate surgical body cavity fluid. By way of non-limiting example, appropriate surgical body cavity fluids include: peritoneal fluid for abdominal, pelvic, or abdominopelvic surgery; pericardial fluid for cardiac surgery; and pleural fluid for thoracic surgery. Post-surgical events that may be monitored also include the incidence of symptoms of post-surgical adhesions: by way of example abdominal, pelvic, or thoracic pain; intestinal obstructions; female infertility; and adhesion of the heart to surrounding tissues. The skilled practitioner may increase the effective amount of APC used in subsequence surgeries, in the same or different subjects, if a further reduction in the incidence of post-surgical adhesions is required.

A therapeutically effective amount or an effective amount of APC may be administered in an appropriate volume of aqueous solution and used to bath the internal organs and tissues as well as the appropriate body cavity, by way of example the peritoneal cavity. An effective amount may be calculated based on the subject's body weight. By way of example, an effective amount may be between about 0.01 and about 1000 micrograms per kilogram of the subject being treated. A preferred effective amount is between about 1 and about 500 micrograms per kilogram of the subject being treated. A more preferred effective amount is between about 10 and about 200 micrograms per kilogram of the subject being treated. Another more preferred effective amount is between about 25 and about 200 micrograms per kilogram of the subject being treated. Another more preferred effective amount is between about 25 and about 100 micrograms per kilogram of the subject being treated. Yet another more preferred effective amount is between about 25 and about 50 micrograms per kilogram of the subject being treated. A most preferred effective amount is about 50 micrograms per kilogram of the subject being treated.

An appropriate volume of aqueous solution is that which would be required to bath the organs and tissues of the body cavity of a particular subject. The appropriate volume of aqueous solution will vary according to the size of the subject, the size of the abdominal, pelvic, or chest incision, and the type of surgery. Surgeries that expose more internal organs will require larger volumes of solution. If desired the surgeon may apply a sterile balanced salt solution to the subject in order to estimate the required or appropriate volume. The appropriate volume of aqueous solution may be determined or estimated, and used to dissolve or dilute the effective amount of APC. APC in the appropriate volume of aqueous solution may then be applied to the internal organs and generally to the appropriate body cavity of the subject.

It is expected that activated Protein C would be administered near the completion of any general, abdominal, pelvic, or chest surgery prior to closing the major incision. An aqueous solution containing an effective amount of APC may be applied to the exposed organs and lining of the particular body cavity after completion of the primary surgery but prior to closing the major incision. Particular attention may be directed to exposed organs and those suspected of receiving mild or significant trauma during surgery. It is anticipated that any or all internal organs may receive treatment, including but not limited to the cecum and rectum, spleen, liver, lungs, reproductive organs, and heart. It is anticipated that the type of surgery may make special attention to certain organs appropriate. By way of non-limiting examples, abdominal surgery may suggest that special attention be paid to organs of the peritoneal cavity. Gastro-intestinal surgery may suggest that special attention be paid to organs of the gastro-intestinal track, including the cecum and rectum. Gynecological surgery may suggest that special attention be paid to the uterus, fallopian tubes, or ovaries. Cardiac surgery may suggest that special attention be paid to the heart and surrounding tissues, including the pericardium. In general, surgeries that involve the manipulation of certain organs and tissues suggest that special attention should be applied to those same organs and tissues, as well as adjacent organs and tissues in order to ensure that they are adequately treated with a solution of APC. Regardless of the type of the surgery, it is recommended that a solution of APC be applied to any site where post-surgical adhesion may develop, and since it is possible that almost any site has the potential to develop post-surgical adhesions, it is preferred that the treatment be applied as extensively as possible.

### III. Subjects and Types of Surgeries

Subjects, as used herein, are meant to include human and non-human mammals. It is expected that human subjects will benefit greatly from the invention. It is also expected that non-human mammalian subjects, including domestic animals, experimental animals, or livestock, will also benefit greatly from the invention. Since all subjects undergoing a general, abdominal, pelvic, abdominopelvic, thoracic, or cardiac surgery may develop post-surgical adhesions, it is anticipated that any subjects undergoing general, abdominal, pelvic, abdominopelvic, thoracic or cardiac surgery may benefit form topical application of APC to the internal organs and tissues. Therefore all subjects undergoing general, abdominal, pelvic, or cardiac surgery are candidates for treatment. It is believed that this method of treating post-surgical adhesions may be applied generally to all surgeries where post-surgical adhesions occur.

It is known that post-surgical adhesions occur following cardiac surgery. Seprafilm has been shown to have a beneficial effect following cardiac surgery in a canine abrasion/desiccation pericardial adhesion model (Seeger et al., (1997) Journal of Surgical Research, 68:63-66). In light of the Inventor's demonstration that the beneficial anti-adhesive effect of APC is significantly greater than HA/CMC in the organs of the abdominal cavity, it is expect that the anti-adhesive effect of APC in preventing post-surgical cardiac adhesion band formation will also be significantly greater than HA/CMC.

The discussion of the references herein is intended merely to summarize the assertions made by the authors and no admission is made that any reference constitutes prior art. Applicants reserve the right to challenge the accuracy and pertinence of the cited references.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLES

The effects of APC were compared to the FDA approved drug sodium hyaluronate/carboxymethylcellulose (HA/CMC, also called Seprafilm). They were compared by administering the two drugs intraperitoneally into 30 male NMRI mice. After 7 days, the pathological adhesion grades were scored by two scaling systems and the concentrations of the pro-inflammatory cytokines, IL-1, IL-6 and TNF-α were evaluated. Inflammation scores of mice in all groups were also measured.

Results:

Relative to HA/CMC group, intraperitoneal administration of APC led to a much higher reduction of adhesion band formation (P<0.05). In the APC-treated group markedly reduced peritoneal fluid concentrations of both IL-6 and TNF-α were also observed. The lowest level of inflammation in adhesive tissues was detected in the APC-treated group.

Conclusions:

These results indicate that in comparison to the FDA approved drug, Seprafilm, APC exhibits a higher efficacy in preventing the postoperative adhesion bands. Thus, APC holds the potential to be therapeutically used as a novel anti-adhesive agent.

### Methods and Materials

Thirty male NMRI mice, weighing from 30 g to 35 g, were randomly divided into 3 groups of 10 mice: group 1, control animals with surgical abrasion and no treatment (n=10); group 2, surgical abrasion plus HA/CMC (Seprafilm, Genzyme Biosurgery Corporation, Cambridge, Mass.) as a gold standard (n=10); group 3, surgical abrasion plus 50 microgram/kg of activated protein C (rhAPC, Dretrecogin alfa{activated}) intraperitoneally (i.p) (n=10). The surgical treatment was performed under a general anesthesia inhalant containing ethoxyethane (ether) and intraperitoneal ketamine hydrochloride (ketamine hydrochloride 50-mg/vial; DeltaSelect; DeltaSelect GmbH, Otto-Hahn-StraRe, Dreiech, Germany) 3 mg/kg. All animal cares and procedures were performed according to the guidelines approved by the ethical committee of Tehran University of Medical Sciences.

Surgical technique and Administration of APC

The Inventors used the adhesion induction model previously described by Hemadeh et al (Hemadeh et al., (1993) Surgery, 114:907-10), which resulted in a 100% occurrence of surgical adhesions in the controls after 7 days. After anesthesia, the skin of abdomen was shaved and cleaned with povidone iodine solution. A vertical midline incision of 2.5 cm was made and abdomen was opened. To induce adhesion band formation, the cecum was exposed, and rubbed gently with 2 dry gauze pads at the ventral and dorsal surfaces until it lost its shine and hemorrhagic points became visible. The cecum was then returned to its anatomic position in abdominal cavity. Before closing the abdominal cavity, the Inventors administered intraperitoneally APC or Seprafilm.

APC was administered as follows. After induction of adhesion bands at the cecum, as described above, a single dose of 50 microgram/kg of APC (Drotrecogin Alfa (Activated)) in 1 ml of saline solution was administered locally to exposed organs in the peritoneal cavity. Particular efferent was made to insure the site of surgery receiving a portion of this topical administration. After application of APC, the abdomen cavity was then closed. Seprafilm was also administered as physical barrier between abdominal wall and exposed organs and also between organs.

After 7 days, mice euthanized and their abdominal cavities were opened by two surgeons who were blinded with regard to previous treatment received by each animal. The mice were scored according to the Nair and Leach scales set forth in Table 1.

Enzyme-linked immunosorbent assay (ELISA) for analysis of cytokines in the peritoneal fluid of experimental mice was conducted using commercial kits and manufactures' instructions.

### Histological Evaluation

After evaluation of the adhesion bands, the membranes and the peritoneal tissues around them were extracted from the other tissues, fixed in 10% formalin and immersed in paraffin. Several Paraffin sections were made by microtome. These sections were stained by Haematoxylin-Eosin (HE) and the degree of inflammation was evaluated using a semi-quantitative scoring system as indicated in Table 1.

TABLE 1
Adhesion and Inflammation Scores Classification. The Table is adopted from Lalountas
(Lalountas et al. (2010) The American Journal of Surgery 200, 118-123).

| Grade | Nair et al* | Leach et al* | Inflammation |
|---|---|---|---|
| 0 | Complete absence of | No adhesion | Nill |
| | adhesions | | |
| 1 | Single band of adhesions, | Filmy thickness, | Giant cells, occasional |
| | between viscera, or from 1 | avascular | scattered lymphocytes |
| | viscus to abdominal wall | | and plasma cells |
| 2 | Two bands: between viscera | Limited vascularity, | Giant cells with |
| | or from viscera to abdominal | moderate thickness | increased numbers of |
| | wall | | admixed lymphocytes, |
| | | | plasma cells, |
| | | | eosinophils and |
| | | | neutrophils |
| 3 | More than 2 bands: between | Well vascularized, | Many admixed |
| | viscera, or viscera to | dense thickness | inflammatory cells, |
| | abdominal wall, or whole of | | micro abscesses |
| | intestines forming a mass | | present |
| | without being adherent to | | |
| | abdominal wall | | |
| 4 | Viscera directly adherent to | — | — |
| | abdominal wall, irrespective of | | |
| | number and extent of | | |
| | adhesive bands | | |

*Refs. (Nair et al., (1974) Arch Surg; 108: 849-53; Leach et al., (1998) Fertil Steril 69: 415-8)

### Statistical Analysis

All variables were expressed as mean±SD and ranks. Differences between the adhesion and inflammation scores of study groups were evaluated by Kruskal-Wallis variance analysis and when significant (P<0.05), the difference between specific mean ranks was determined using the multiple comparison procedure based on ranks. The differences between ELISA samples were also evaluated by one-way Anova test. The level of significance was set to 5%.

### Example 1

### Grade of Adhesion Bands

The grades of post-surgical intraperitoneal adhesion bands (frequency and distribution) in experimental mice were measured after 7 days according to two methods described by Nair (Nair et al., (1974) Arch Surg; 108:849-53) and Leach (Leach et al., (1998) Fertil Steril 69:415-8) (FIG. 1). There were no mortalities due to anesthetic induction or surgical procedures. No serious complications, such as bleeding related to the dosage of APC required to reduce adhesion formation, was detected. The grades of adhesion bands (frequency and distribution) were measured and the Nair et al scaling method was applied as presented in Table 2. The experimental animals with surgical abrasion which received only the vehicle, showed results similar to the control group (data not shown). The adhesion score (mean±SD) in group 1 (untreated group) was 2.9±0.737; in group 2 (Seprafilm), 1.4±0.843; in group 3 (APC), 0.4±0.699. Significant differences were found between the mean adhesion score in groups 1 and 2 relative to untreated control group (P<0.05) (FIG. 2). There were also significant decreases in mean adhesion scores in group 3 when compared to group 2 (P<0.05).

It should be noted that the experimental animals with surgical abrasions receiving only the vehicle (normal saline) showed results similar to those observed in the control group (data not shown). There were no mortalities due to the anesthetic use or surgical procedures in any group. Furthermore, no significant complication such as bleeding related to the dose of APC, required to reduce adhesion band formation, was detected in any of the experimental animals. The efficacy of several different concentrations of APC (10, 25, 50, 100 and 200 μg/kg) was evaluated. The APC concentration of 25 μg/kg was determined to be only slightly less active than the 50 μg/kg dose which yielded the maximal protective effect in preventing adhesion band formation (data not shown). Increasing the concentration of APC above 50 μg/kg did not result in any further protective effect. Taken together, the results presented in FIG. 2 clearly suggest that APC is markedly more effective than Seprafilm in preventing post-surgical intraperitoneal adhesion band formation.

The frequencies (Table 2) and grades of adhesion bands (FIG. 3) were also measured according to Leach et al. scaling. Similar outcomes were observed for the grades of adhesion bands scored by the two methods described above.

TABLE 2
Frequency and distribution of adhesion scores in mice.

| | Group 1 | | Group 2 | | Group 3 | |
|---|---|---|---|---|---|---|
| Score | Nair | Leach | Nair | Leach | Nair | Leach |
| 0 | 0 | 0 | 2 | 2 | 7 | 7 |
| 1 | 0 | 2 | 2 | 4 | 2 | 3 |
| 2 | 3 | 4 | 6 | 4 | 1 | 0 |
| 3 | 5 | 4 | 0 | 0 | 0 | 0 |
| 4 | 2 | — | 0 | — | 0 | — |
| Mean ± | 2.9 ± | 2.2 ± | 1.4 ± | 1.2 ± | 0.4 ± 0.699 | 0.3 ± 0.483 |
| SD | 0.737 | 0.788 | 0.843 | 0.785 | | |

### Example 2

### Histological Evaluation

The inflammation score of untreated control group was dominant at serosal surface of cecum. After 1 week, peritoneal surface in mice was surrounded by inflammatory cells such as giant cells, neutrophils, plasma cells and lymphocytes (FIG. 4). Histology of peritoneal tissues in two magnifications for the untreated control group (A), the Seprafilm-treated group (B) and the APC-treated group (C) are presented in FIG. 4. In the untreated control group, many inflammatory and giant cells were found to be recruited to the peritoneal tissues and some micro abscesses could also be observed. The same cell types, but to a lesser extent, could also be detected in the photomicrographs of the Seprafilm-treated animals. In contrast to both the control and Seprafilm groups, the number of inflammatory cells were markedly decreased and essentially no micro abscesses could be observed in the APC-treated group (FIG. 4, panel C). There were only a few scattered leukocytes in the APC-treated groups.

The inflammation grades in all groups were evaluated (FIG. 5). The mean±SD of inflammation scores determined by two pathologists in a blind-study on a scale of 0-3 (Dinarvand et al., (2012) J Surg Res. 2012; 172(1):el-9; Hooker et al., (1999) Surgery. 125(2):211-216) were: 2.4±0.516 in the untreated control group, 1.6±0.699 in the Seprafilm group, and 0.5±0.849 in the APC group. Statistically significant differences were observed between mice receiving either Seprafilm or APC and those receiving no treatment (p<0.05). In comparing the treated groups (APC and Seprafilm), a significant decrease of the inflammation score was observed for the APC group (FIG. 5). The significantly lower inflammation score of APC is consistent with its dramatic inhibitory effect on the expression of proinflammatory cytokines as demonstrated above in FIGS. 6 through 9.

### Example 3

### Analysis of Pro-Inflammatory Cytokines

The peritoneal fluid concentrations of IL-1, IL-6 and TNF-α, determined by ELISA, are presented in FIGS. 6, 7, and 8, respectively. The peritoneal fluid concentrations of both IL-6 (FIG. 7) and TNF-α (FIG. 8) were significantly reduced in the APC-treated group. The expression levels of cytokines were measured at four time points of 0 h, 6 h, 24 h and one week post-surgery At the 6 h time point following the surgery, the concentration of IL-1 was elevated greater than 6-fold in the control group and the treatment with either Seprafilm or APC resulted in a modest but statistically significant decrease in the cytokine level (FIG. 6). However, no statistically significant differences in the IL-1 levels were observed between the three groups after 24 h following surgery, though the cytokine level gradually declined and reached near the baseline level one week post-surgery (FIG. 6). By contrast, the concentration of IL-6 was elevated in both the control and Seprafilm-treated groups, however, its expression was markedly inhibited in the APC-treated group after 6 h following surgery (FIG. 7). The IL-6 level was decreased to a baseline level in the APC-treated group 24 h post-surgery. On the other hand, the expression level of IL-6 was not affected by Seprafilm and remained elevated for the entire duration of the experiment (FIG. 7). Similarly, APC effectively inhibited the expression level of TNF-α, thus decreasing it to a near baseline level at all three time points examined (6 h, 24 h and one week) (FIG. 8). By contrast, TNF-α remained high in both untreated control and Seprafilm groups and its level was only modestly decreased by Seprafilm one week post-surgery (FIG. 8).

### Example 4

The peritoneal fluid concentrations of TGF-β concentration was monitored for up to 24 h. While Seprafilm had no effect on the expression of TGF-β, APC dramatically inhibited its expression at both the 6 h and 24 h time points (FIG. 9). The induction of TGF-β expression following surgery was rapid since by the time the experiments were completed and the peritoneal fluid sampling of the animals for the first time point was initiated (−15-20 min), the level of TGF-β was already markedly elevated (FIG. 9). This was not surprising since TGF-β is a profibrotic cytokine, playing an essential role in the wound healing process following injury (Chegini (2008) Semin Reprod. Med. 2008; 26(4):298-312.). Thus, the 0 h time point does not accurately reflect the baseline TGF-β in these animals. For determining the baseline TGF-β level, we had to take peritoneal fluid samples from four additional mice without subjecting them to any surgical procedures (these data are presented in FIG. 9). Analysis of these data in FIG. 9 clearly suggests that TGF-β is secreted rapidly after surgery. By contrast to Seprafilm, APC potently inhibited the induction of TGF-β in this injury model. Moreover, APC exhibited its protective activity early after administration since a significant decrease (p <0.05) in the concentration of the peritoneal TGF-β was observed in the APC-treated group at the first time point after surgery (labeled 0 h in FIG. 9).

### Example 5

The Inventors measured the concentration of tPA in the intraperitoneal fluids of experimental animals and noted that the level of this fibrinolytic enzyme is markedly elevated in the APC-treated group 6 h post-surgery (FIG. 10). Taken together, results clearly demonstrate that APC is much more potent than Seprafilm in enhancing fibrinolysis and inhibiting the post-surgical proinflammatory responses. The Inventors believe these anti-inflammatory and profibrinolytic properties of APC are primarily responsible for its ant adhesive property in the experimental animals.

Conclusion. Taken together, these results indicated that APC is much more potent than Seprafilm in preventing post-surgical adhesion band formation. Thus, it is believed that APC could be used as a novel therapeutic anti-adhesive drug for post-surgical adhesion band formation as it occurs in all internal organs after general, abdominal, pelvic, or cardiac surgeries.

All publications and patents cited in this specification are hereby incorporated by reference in their entirety. The discussion of the references herein is intended merely to summarize the assertions made by the authors and no admission is made that any reference constitutes prior art. Applicants reserve the right to challenge the accuracy and pertinence of the cited references.

What is claimed is:

1. A method of reducing the incidence of post-surgical adhesions in a subject undergoing surgery, the method comprising,
a) completing a primary surgery,
b) topically administering an effective amount of activated protein C to the organs and tissues exposed by the primary surgery or subjected to surgical manipulation by the primary surgery, and
c) closing a major incision associated with the primary surgery.

2. The method of claim 1, wherein the effective amount is about 25 micrograms per kilogram to about 200 micrograms per kilogram of the subject being treated.

3. The method of claim 1, wherein the effective amount is about 25 micrograms per kilogram to about 100 micrograms per kilogram of the subject being treated.

4. The method of claim 1, wherein the effective amount is about 25 micrograms per kilogram to about 50 micrograms per kilogram of the subject being treated.

5. The method of claim 1, wherein the effective amount is about 50 micrograms per kilogram of the subject being treated.

6. The method of claim 1, wherein the surgery is abdominopelvic surgery.

7. The method of claim 1, wherein the surgery is abdominal surgery.

8. The method of claim 1, wherein the surgery is pelvic surgery.

9. The method of claim 1, wherein the surgery is cardiac surgery.

10. The method of claim 1, wherein the surgery is thoracic surgery

11. The method of claim 1, wherein the subject exhibits reduced symptoms of post-surgical adhesions.

12. The method of claim 1, wherein the subject exhibits reduced indicators of post-surgical inflammation as measured in the surgical body cavity fluid.

13. The method of claim 12, wherein reduced indicators of post-surgical inflammation are selected from the group consisting of Il-1, Il-6, TNFα, and TGFβ.

14. The method of claim 1 wherein the subject exhibits reduced tPA in the surgical body cavity fluid

15. The method of claim 1 wherein activated protein C consist of Drotrecogin Alfa Activated.

16. The method of claim 1 wherein the activated protein C consist of SEQ ID NO:1, containing conservative amino acid substitutes, secreted from an eukaryotic cell, and activated in vitro, wherein the activated protein C possesses substantially full APC activities.

17. The method of claim 1 wherein activated protein C consist of SEQ ID NO:1 secreted from an eukaryotic cell, and activated in vitro.

18. The method of claim 1 wherein the subject is a human subject.

19. The method of claim 1 wherein the subject is a non-human mammalian subject.

20. A method of reducing the incidence of post-surgical adhesions in a human subject undergoing abdominal surgery, the method comprising,
a) completing a primary surgery,
b) topically administering about 50 micrograms per kilogram of the human subject, of activated protein C, to the organs and tissues exposed by the primary surgery or subjected to surgical manipulation by the primary surgery, and
c) closing a major incision associated with the primary surgery.
